(19) United States
(12) Patent Application Publication
(10) Pub. No.: US 2008/0114452 A1
(43) Pub. Date: May 15, 2008

(54) PROSTHESIS EXHIBITING POST-IMPLANTATION SIZE CHANGE

(76) Inventor: Shlomo Gabbay, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/939,788

(22) Filed: Nov. 14, 2007

Related U.S. Application Data
(60) Provisional application No. 60/858,798, filed on Nov. 14, 2006.

(51) Int. Cl. A61F 2/24

(52) U.S. Cl. 623/2.42; 623/2.17

(57) ABSTRACT

An expandable type of heart valve prosthesis includes means for retaining the prosthesis substantially at a first cross-sectional dimension that is smaller than a second, expanded cross-sectional dimension, and for breaking down to permit the support structure to expand toward the second larger cross-sectional dimension after being implanted for a period of time.

### RELATED APPLICATION

This application claims the benefit of U.S. provisional patent application No. 60/858,799 which was filed Nov. 14, 2006 entitled PROSTHESIS EXHIBITING POST-IMPLANTATION SIZE CHANGE, the contents of which is incorporated herein by reference.

### TECHNICAL FIELD

The present invention relates generally to implantable cardiac apparatuses and, more particularly, to a prosthesis capable of changing its size post-implantation.

### BACKGROUND

Congenital heart abnormalities, in the absence of appropriate surgical treatment, often result in an extremely poor life expectancy and/or quality of life. One particular abnormality is commonly referred to tetralogy of Fallot, which causes anatomic variability in the pulmonary outflow tract and pulmonary arteries. Some anatomic variations stemming from tetralogy of Fallot appear at infancy, such as stenosis at the pulmonary annulus, which may be associated with hypoplasia of the main pulmonary artery. Such anatomic variations may cause severe hypoxemia. Other anatomic variations associated with tetralogy of Fallot may not manifest symptoms until later in childhood, which may include infundibular stenosis with mild or no stenosis at the pulmonary arteries or branch pulmonary arteries.

If tetralogy of Fallot is left untreated, the pulmonary artery system may not develop sufficiently to accommodate total cardiac output, which is due to closure of the ventricular septal defect. This often causes right ventricular failure and mortality resulting from low cardiac output.

Several surgical techniques have been developed to help repair obstructions of the right ventricular outflow tract (RVOT), such as may be associated with tetralogy of Fallot and other conditions. These may include palliative procedures and total correction, depending on the particular circumstances associated with the patient's condition. Though it is most common for total correction to be utilized. In an effort to alleviate pulmonary insufficiency, some surgeons utilize a patch that has a pericardial cusp formed thereon as part of RVOT reconstruction. The cusp, which is usually formed during the surgical procedure by fixation of the pericardium in a glutaraldehyde solution, is provided to compensate for the damaged cusp(s). This approach is not completely satisfactory as the cusp typically does not last.

In other situations surgeons may implant a heart valve. However, when a heart valve is implanted in an infant, toddler or other younger patient, re-operation is usually required on several occasions so that the implanted valve is properly sized to the patient's heart. In order to surgically implant each heart valve into the patient, the patient typically is placed on cardiopulmonary bypass during a complicated, but common, open-chest and open-heart procedure.

### SUMMARY

The present invention relates to a prosthesis that can change size post-implantation. For example, a heart valve prosthesis according to an embodiment of the present invention can be implanted in an infant or small child with a first cross-sectional dimension. As the child grows, the heart valve prosthesis can expand to a second larger cross-sectional dimension commensurate with the growth of the patient's heart.

One aspect of the present invention provides a heart valve prosthesis that includes a generally cylindrical support structure configured to expand from a first cross-sectional dimension to a second cross-sectional dimension, which is larger than the first cross-sectional dimension. A valve is attached to the support structure to provide for substantially unidirectional flow of blood through the prosthesis in a direction from an inflow end to an outflow end thereof. A biodegradable member is operatively connected with the prosthesis to maintain temporarily the support structure at a cross-sectional dimension that is smaller than the second cross-sectional dimension. As a result, after being implanted for a substantially predetermined time period, the biodegradable material breaks down to permit the support structure to expand toward the second larger cross-sectional dimension.

Another aspect of the invention provides an expandable type of heart valve prosthesis. The prosthesis includes means for retaining the heart valve prosthesis substantially at a first cross-sectional dimension that is smaller than a second, expanded cross-sectional dimension, and for breaking down to permit the support structure to expand toward the second expanded cross-sectional dimension after being implanted for a substantially predetermined time period. The prosthesis also includes means for expanding the prosthesis from the first cross-sectional dimension toward the second expanded cross-sectional dimension.

### BRIEF DESCRIPTION OF THE DRAWINGS

FIG. 1 depicts a partial assembly view of an example prosthesis that can be implemented according to an embodiment of the invention.

FIG. 2 depicts an example of a prosthesis that can be implemented according to an embodiment of the invention.

FIG. 3 depicts part of the apparatus of FIG. 2 illustrated in a second condition according to aspect of the invention.

FIG. 4 depicts the prosthesis of FIG. 2 being manually expanded according to an aspect of the invention.

FIG. 5 depicts an end view of a prosthesis in an expanded condition according to an aspect of the invention.

FIG. 6 depicts a side view of a prosthesis in an expanded condition according to an aspect of the invention.

FIG. 7 depicts an example of a prosthesis implanted in a heart according to an aspect of the invention.

FIG. 8 depicts an example of a prosthesis implanted in a heart in an expanded condition according to an aspect of the invention.

FIG. 9 depicts an example of a second prosthesis implanted in a previously implanted and expanded prosthesis according to an aspect of the invention.

### DETAILED DESCRIPTION

The invention relates generally to a prosthesis that can change size or cross-sectional dimensions after implantation.

As one example, the heart valve prosthesis includes a sidewall portion that extends between inflow and outflow ends of the prosthesis. A support structure is configured to expand from a first, smaller cross-sectional dimension to a second, larger cross-sectional dimension. The expansion can be automatic (e.g., self-expanding) or it can be implemented manually, such as by applying an expansion force to the support structure. A valve is located within the sidewall portion of the support to provide for substantially unidirectional flow of blood from the inflow end to the outflow end. A member is operatively connected with the prosthesis to maintain the support structure (and the prosthesis) at a cross-sectional dimension that is smaller than the second, larger cross-sectional dimension to which the support structure can be expanded. The member comprises a biodegradable material whereby, after being implanted for a substantially predetermined time period, the biodegradable material breaks down to permit the support structure to expand the prosthesis toward the second larger cross-sectional dimension. For example, the biodegradable material can be implemented as absorbable sutures, biocompatible (but non-fixed) tissue material, biodegradable synthetic materials or a combination thereof.

The prosthesis is beneficial for treating defects and other conditions for infants or small children since the prosthesis can expand from a cross-sectional dimensions at implantation to a larger size as the patient grows. As a result, re-operation can be mitigated or at least reduced for a significant population of patients requiring replacement heart valves.

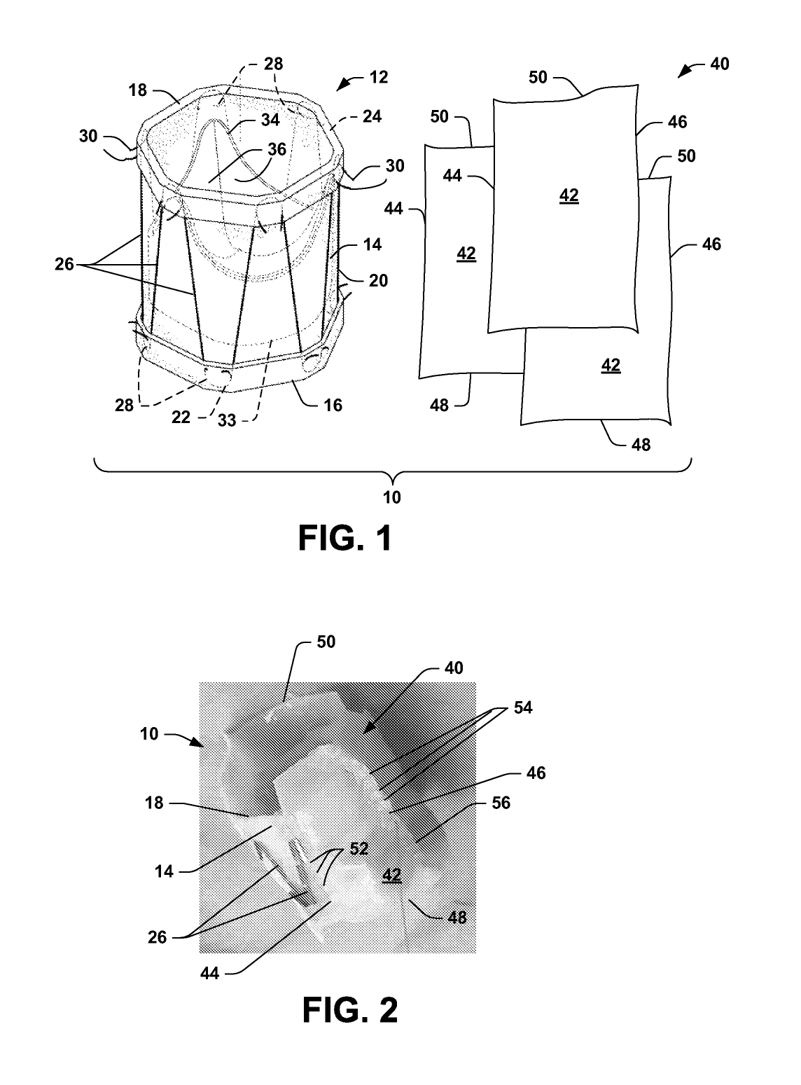

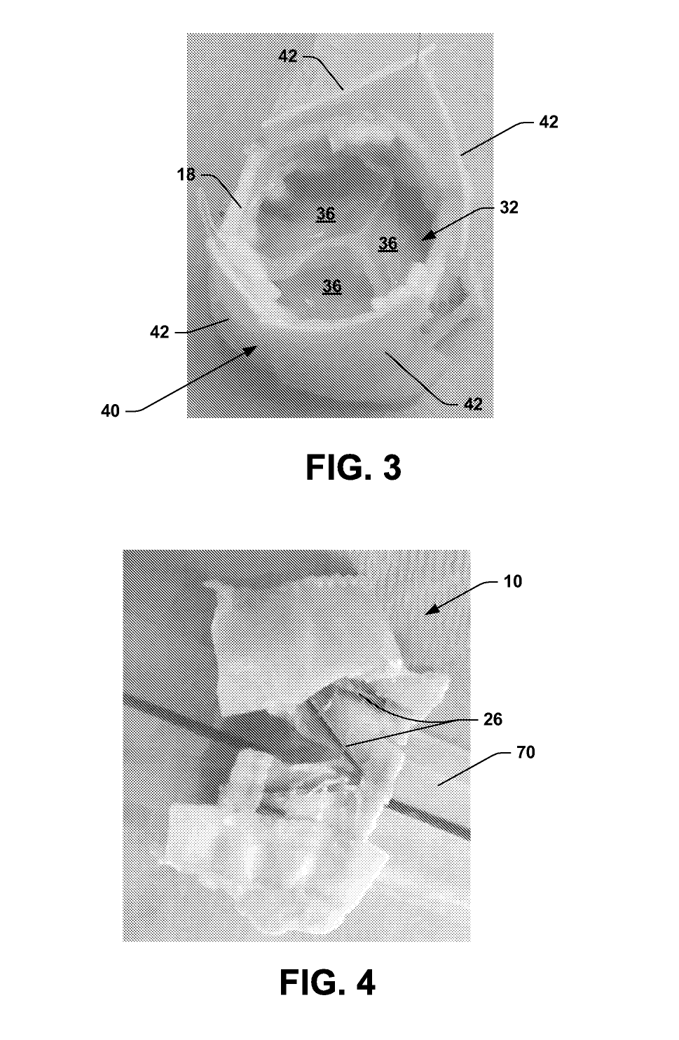

FIG. 1 depicts an example of one embodiment of a heart valve prosthesis system 10 that can be implemented according to an aspect of the invention. FIGS. 2 and 3 illustrate assembled and partially assembled embodiments of the prosthesis 10. The prosthesis 10 includes a prosthetic valve 12 that includes a sidewall portion 14 that extends between an inflow end 16 and an outflow end 18. The prosthetic valve 12 includes a support that is expandable radially outwardly. By expandable, it is meant that the support 20 can expand from a reduced cross-sectional dimension (e.g., diameter) to a larger cross-sectional dimension. The expansion can be automatic, such that the support 20 is self-expanding, or the expansion can be manual, such that some force may be needed to implement the expansion. For example, the expansion can be implemented by placing a balloon catheter (or other structure) at an interior of the support and causing the balloon to expand and engage an interior of the support or the valve 12 mounted therein.

The support 20 includes axially spaced apart ends 22 and 24 interconnected by generally axially extending support features 26. In the example of FIG. 1, adjacent support features 26 are interconnected by arcuate junctures 28 at the respective ends 22 and 24 so as to define a generally sinusoidal or zig-zagging sidewall portion thereof arranged in a generally cylindrical configuration between the ends. In the example of FIG. 1, there are six junctures 28 at each of the respective ends 22 and 24 that are interconnected by associated support features 26, which can be an integral support structure (e.g., monolithic).

The end junctures 28 in the example of FIG. 1 are curved and operate as self-biasing springs that provide for self-expansion of the support by urging adjacent pairs of interconnected support features apart from each other. Alternatively, self-expansion can be a material property (e.g., memory) of the support 20. Those skilled in the art will understand and appreciate that other numbers (e.g., 2, 3, 9, 12 and so forth) and configurations of end junctures 28 can be utilized at each end. It is further to be appreciated that other types and configurations of expandable support structures or stents can also be utilized. For example, as an alternative to curved interconnecting end junctures 28 shown in FIG. 1, such ends could be pointed or rectangular.

The support 20 can also include one or more projections or spikes 30 that extend axially and radially outwardly from at least some of the respective end junctures 28 of the support. While a pair of such spikes 30 is illustrated as associated with each end juncture 28, other number of spikes can be implemented, such as single spike or more than two spikes at some or all of the junctures. The spikes 30 at opposite ends can be oriented toward the opposing ends 18, 16 to mitigate movement in different directions, such as by having each spike 30 forming an acute angle relative to its associated support feature 26 from which it extends. Alternatively, such spikes could be omitted from the prosthesis 10.

By way of further example, the support 20 can be formed a shape memory material, such as NITINOL. As an example, the support can be formed as from a small cylindrical tube of the shape memory material, such as via a laser cutting (ablation) process in which the desired shaped sidewall is cut from the tube. In this way, the support features 26, the interconnecting end junctures 28, and associated spikes 30 can be formed as an integral structure (e.g., monolithic or a single piece) structure having a desired shape and size. Other processes (e.g., injection molding, casting and the like) can also be used to make the support 20. Additionally, ends of the spikes 30 can have tapered or sharpened tips to facilitate gripping surrounding tissue when implanted. For example, the spikes 30 can be formed by laser cutting from the same tube or, alternatively, they could be welded onto the support 20 at desired positions. The resulting structure can then be heated to its transformation temperature and forced to a desired cross-sectional dimension and configuration (its austentic form). The support 20 can then be bent or deformed to a reduced cross-sectional dimension when in its low-temperature (martensitic) form to facilitate its mounting within a barrel of an implanter, for example.

The prosthesis 10 also includes a heart valve 32 mounted within the support 20. The valve 32 includes an inflow end 33 and an outflow end 34 at axially opposed ends of the valve, with a sidewall portion of the valve extending between the ends thereof. The inflow end 33 of the valve 32 is positioned near an inflow end 22 of the support 20. The cross-sectional dimension of the prosthesis 10 can vary according to the cross-sectional dimension of the support 20. That is, the prosthetic valve 32 can expand from a reduced cross-sectional dimension to an expanded condition as described herein. The valve 32 can be dimensioned to provide for a sufficient amount of coaptation between adjacent leaflets 36 thereof over a range of cross-sectional dimensions.

For example, the valve 32 can operate to provide for substantially unidirectional flow of blood through the prosthesis at first, cross-sectional dimension as well to provide for substantially unidirectional flow of blood at a second, larger cross-sectional dimension (as well as for sizes between the first and second dimensions). This can be achieved by oversizing the valve 32 for the smaller first cross-sectional dimension. As one example, a valve having an normal diameter of about 25-30 mm diameter can be compressed within the support 20 to a 12-17 mm diameter for an initial period of use, as described herein. After this initial period, the support 20 can expand (or be expanded by a minimally invasive technique) the valve 32 commensurate with growth of the patient's heart and, more specifically, the anatomical site where the valve is implanted. It is to be understood and appreciated that other deformable, expandable types of supports can also be utilized in accordance with an aspect of the invention, which can include self-expanding supports and manually expandable supports.

As a further example, the sidewall portion 14 of the prosthesis can be a tubular valve wall of the valve 32, such as if the valve is implemented as a homograft or xenograft valve. Continuing with this example, the valve 32 can include a one or more (e.g., a plurality of) leaflets 36 that extend radially inward from the sidewall and coapt along their adjacent edges to provide for substantially unidirectional flow of blood through the valve 32 from the inflow end to the outflow end. The outflow end 34 of the valve 32, which is located near the outflow end 24 of the support 20, can have a generally sinusoidal contour or it could be substantially flat at the end. The peaks (corresponding to commissures of the valve leaflets 36) can be aligned generally with and attached to support junctures 28 at the end 24 of the support 20. The valve 32 can be connected within the support 20 via sutures or other known means of attachment, for example, clips, hooks, adhesive materials or the like.

It is to be understood and appreciated that various types of valve configurations of could be employed to provide the prosthesis 10 in accordance with an aspect of the invention. For example, as mentioned above, the valve 32 can be a homograft or xenograft. Alternatively, the valve 32 can be manufactured from natural or synthetic materials or from a combination of natural and synthetic materials and include one or more leaflets. By way of further example, the valve 32 can be a treated pulmonic valve (e.g., homograft or xenograft), the valve 32 can be a treated aortic valve (e.g., homograft or xenograft), as well as others mentioned herein or otherwise known in the art.

The prosthesis 10 can also include a retaining member 40 configured to maintain the support and the prosthesis at a cross-sectional dimension that is smaller than a second cross-sectional dimension to which the prosthesis can expand. The retaining member 40 includes a biodegradable material such that, after being implanted in a patient for a substantially predetermined period of time, the biodegradable material breaks down to permit the support structure and the valve mounted therein to expand toward the larger cross-sectional dimension. Thus, the retaining member 40 temporarily retains the prosthesis 10 with its reduced cross-sectional dimension after being implanted.

The expansion can occur automatically after a sufficient amount of the retained mechanism has been broken down to enable self-expansion of the prosthesis to a larger cross-sectional dimension. Alternatively, the expansion can be implemented manually such as by inserting a balloon catheter or other mechanism that can urge the support radially outwardly toward the larger second cross-sectional dimension. The approach can vary, for example, based on the type and elasticity (or memory) of the material that is used to form the support 20. It will further be appreciated that by providing the retaining member to maintain the cross-sectional dimension at a reduced diameter for an initial period of time, healing is promoted at the implantation site.

As used herein, the term “biodegradable” and variants thereof refers to the capability of being decomposed (or broken down) by natural biological and/or chemical processes such as may occur in or around a patient's heart. Those skilled in the art will understand and appreciate various types of biodegradable materials that can be utilized to form at least a portion of the retaining member 40. For example, the retaining member 40 can be formed of one or more sheets of a natural or synthetic material, absorbable sutures, or a combination thereof. As one example, such sutures can be absorbable synthetic (e.g., polyglycolic acid and polyglactin) sutures or absorbable natural (e.g., chromic catgut) sutures. Additionally, or alternatively, the retaining member 40 can be formed of one or more sheets of a soft, pliant material, such as animal pericardium, dura matter, fascia lata and the like that, but treated to render it biocompatible yet absorbable (e.g., not fixed in a fixation solution, such as is often done with glutaraldehyde), such that the sheet(s) of material will break down after a period of time. Thus, one or more component parts of the retaining member can be biodegradable over time as to provide means for retaining the prosthesis in a reduced cross-sectional condition for an initial period and after breaking down sufficiently, permit expansion of the prosthesis toward an expanded cross-sectional condition.

In the example of embodiment shown in FIGS. 1-3, the retaining member 40 is formed of a plurality of sheets 42 of a tissue material that is attached around a compressed the heart valve 12 while in a reduced cross-sectional dimension. In the particular example embodiments of FIGS. 1-3, the retaining member 40 includes a plurality (e.g., three) sheets 42 of a pliable tissue material, although other numbers of sheets (e.g., 1, 2, 4, 5 etc.) can be used. Each of the sheets 42 has respective side edges 44 and 46 that extend between ends 48 and 50. In this example, each of the sheets 42 are of similar dimensions, although differently sized sheets could also be utilized. The sheets 42 cam be applied around the sidewall 14 of the prosthesis 10 to cover the exterior as well as be attached to the prosthesis 10 by absorbable (e.g., biodegradable) sutures.

For example, as shown in FIG. 2, one edge 44 is sutured to the sidewall of the prosthesis and another edge 46 can be sutured to the edge 44 of a different sheet to create an axially extending flange 56 formed by the commissure of the two edges 44 and 46 by the absorbable sutures. The other edges of the respective sheets 42 of the material can be attached in a similar way around the exterior of the valve such that the sheets, after being attached together via the sutures, maintain the prosthesis 10 in a reduced cross-sectional dimension. The edges that are sutured together to form the respective flanges 56 can be sutured together at the time of implantation or, alternatively, the edges can be sutured in advanced by the manufacturer of the prosthesis. After a substantially predetermined period of time of being implanted (e.g., two to three months), the biodegradable portions of the retaining member 40 (e.g., absorbable sutures in the embodiments of FIGS. 1-3) are sufficiently absorbed or biodegraded, the implanted prosthesis 10 can begin expanding commensurate with the growth of the implantation site in the patient's heart. To facilitate expansion of the valve, the folded portion of the sidewall 14 intermediate pairs of adjacent connecting members 28 can be cut axially, such as just prior to implantation. In this way, the prosthesis can expand beyond the dimensions typically permitted by the sidewall 14 of the prosthesis 10.

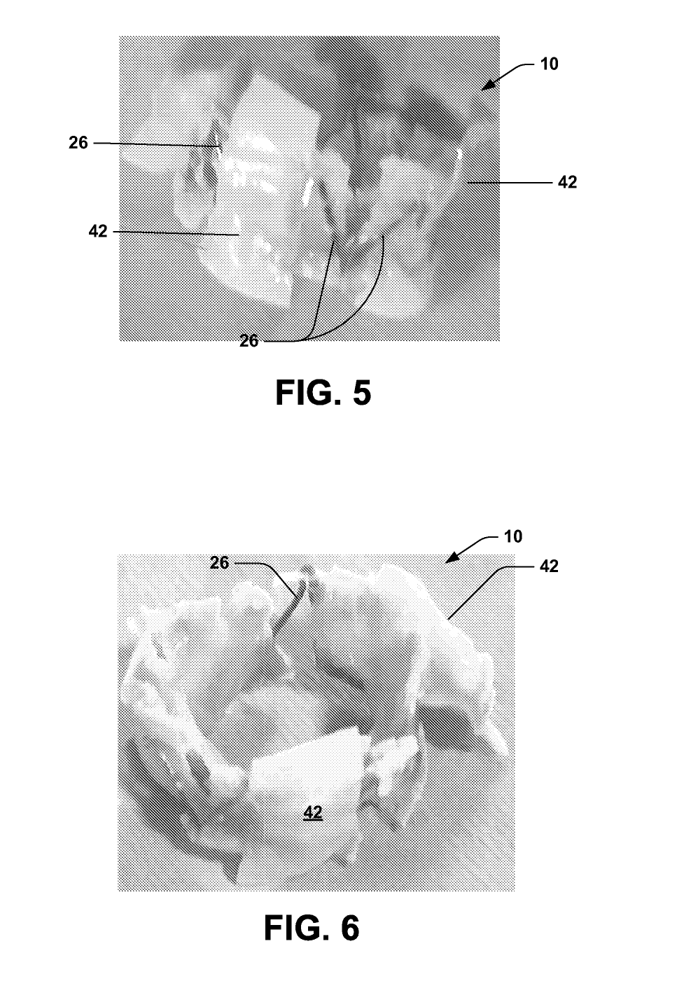

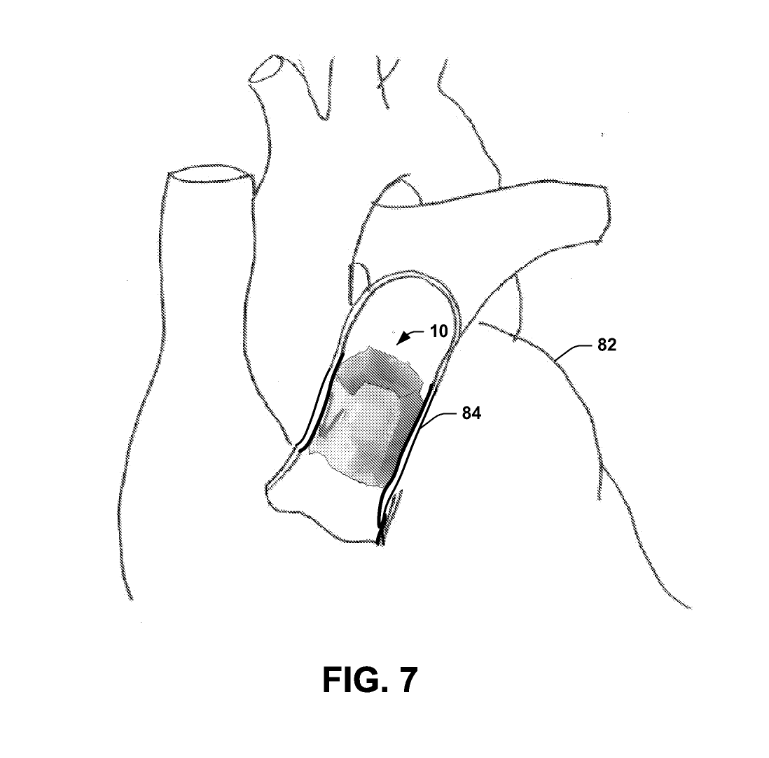

If and when the prosthesis 10 becomes insufficient, the prosthesis can be dilated further such as via use of a balloon catheter 70, such as is shown in the example of FIG. 4. After the prosthesis 10 has been dilated to an appropriate size, another valve (not shown) can be implanted directly within the dilated, enlarged prosthesis 10. Since the original heart valve prosthesis can be dilated and remain in place without requiring its removal, a non-invasive technique can be utilized to dilate the prosthesis and to implant the new heart valve prosthesis with little or no cardio pulmonary bypass. Advantageously, the new valve can be implanted directly within the original valve as to provide an appropriate diameter according to the amount of dilation and size of the patient's heart and of the implantation site within the prosthesis 10. Alternatively, the insufficient prosthesis can be removed prior to implantation of the new heart valve prosthesis. However, the FIGS. 5 and 6 illustrate different views of a prosthesis 10 in a fully expanded condition such as after dilation with the balloon catheter 70 (as shown in FIG. 4). Thus, as shown in FIGS. 5 and 6, the individual sheets 42 of the retaining member 40 remain attached to the exterior of the prosthesis but the attachment via the absorbable sutures to other adjacent sheets no longer exists due to the absorption or breakdown of the sutures utilized to attach to the sheets together.

By way of further example, since the prosthesis 10 is expandable, the prosthesis can be implanted utilizing non-invasive techniques, such as shown and described in co-pending application Ser. No. 10/266,380 which is entitled HEART VALVE PROSTHESIS AND SUTURELESS IMPLANTATION OF A HEART VALVE, which is incorporated herein by reference. While the implantation can be considered sutureless, one or more sutures can also be applied to further secure the prosthesis relative to the implantation site within the expanded prosthesis 10 in the patient's heart.

While in the example shown and described with respect to FIGS. 1-6, the sutures have been described as being absorbable, it will be understood and appreciated that other types of retaining members and other materials can be utilized as forming the biodegradable portion of the retaining member 40. For instance, the sheets themselves can be biodegradable, such as by not fixing the material in an aldehyde or other fixation solution. Other synthetic and naturally occurring material may also exhibit sufficient biodegradable properties to be useful in providing the retaining member 40 for the prosthesis 10. As yet another alternative, absorbable sutures can be utilized in the absence of the sheets of material such as the sutures can be wound circumferentially in multiple loops around the prosthesis to maintain the prosthesis in its reduced cross-sectional dimension at implantation temporarily (until being sufficiently absorbed) and subsequently thereafter be absorbed to permit further expansion of the prosthesis. Additionally, it will be understood that the time period at which biodegradation occurs sufficiently to enable the expansion of the prosthesis 10 can vary according to the amount and types of materials utilized as the retaining member. Thus, the post-implantation timing at which expansion occurs automatically can be considered a controlled parameter based on properties of the materials utilized to form the retaining member 40. For example, the time period can be set to provide for expansion of the prosthesis at least four weeks after implantation. In other situations, two months (or longer) maybe a desirable time period to postpone the expansion of the valve. As described herein, the expansion can be automatic or manual means can be utilized to expand the prosthesis.

By employing a cardiac prosthesis that will permit post-implantation expansion of the valve, the prosthesis 10 is maintained in its reduced cross-sectional dimension to promote regular healing of the patient and to facilitate insemination into the body. After the sutures or other biodegradable portion(s) of the retaining member 40 has been appropriately absorbed by the patient's body, the prosthesis 10 can expand commensurate with the growth of the child and the child's heart without requiring immediate re-operation as with many existing approaches. As mentioned above, once stenosis or regurgitation has been observed, the prosthesis 10 can be further expanded, such as by balloon catheterization, and a new valve can be injected concentrically within the prosthesis, such as a valve is shown and described in the above incorporated co-pending U.S. patent application Ser. No. 10/266,380.

By way of further example, FIGS. 7-9 depict part of a procedure that can be employed to expand or dilate an implanted prosthesis 10 and implant a second, larger heart valve prosthesis 80 into a patient's heart 82 at substantially the same site as the initial prosthesis 10. The procedure can be performed during an open or closed chest procedure. Additionally, if an open chest procedure is performed, the second valve 82 can be implanted during a low-invasive closed heart procedure.

FIG. 7 depicts the heart valve prosthesis 10 implanted in the pulmonic position (in the outflow tract of the pulmonary artery 84). At this stage, it is assumed that the retaining member has already biodegraded and the valve has expanded to or beyond its intended expanded condition. For example, the valve prosthesis 10 may not be sufficient or regurgitation may have been observed or some other condition indicates an insufficiency of the prosthesis 10. Due the configuration of the prosthesis 10, as described herein, the prosthesis can remain implanted and the second valve 80 can be implanted within the original prosthesis.

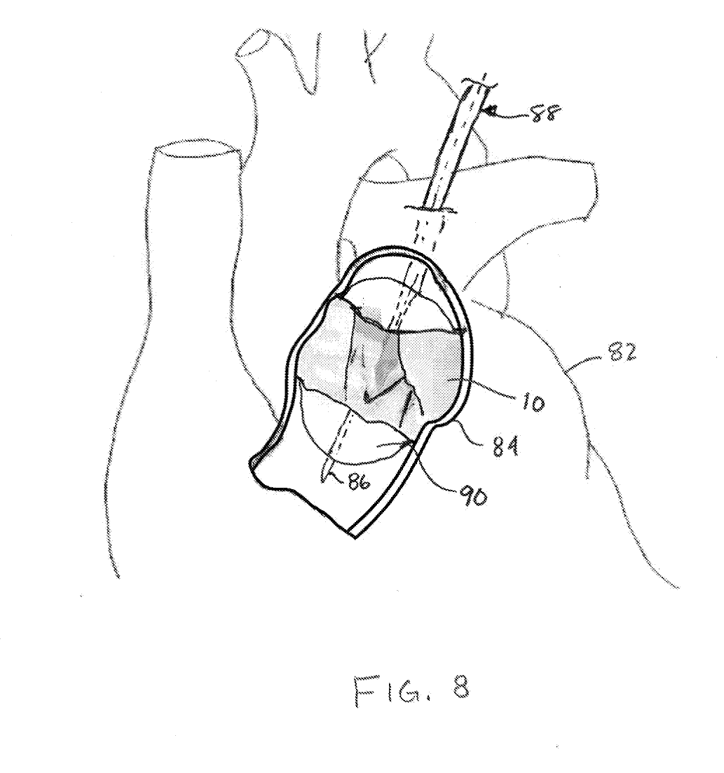

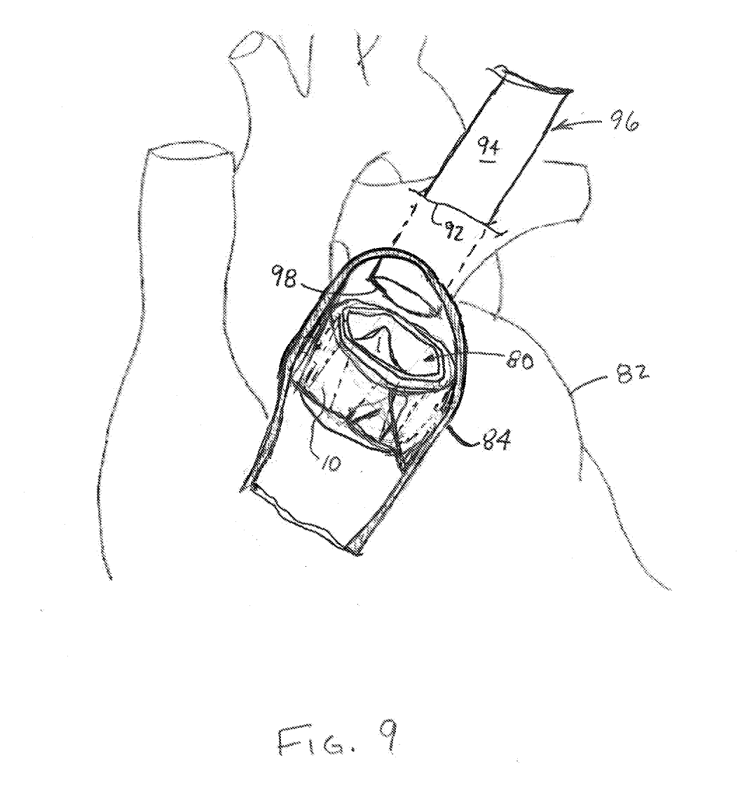

FIG. 8 depicts an example of the prosthesis 10 being expanded according to one aspect of the invention. For instance, a distal end 86 of a catheter 88 can be guided into an interior of the prosthesis 10. The catheterization process can be assisted by an imaging device (e.g., fluoroscopy, ultrasound, x-ray or the like) as is known in the art. A balloon 90 located adjacent the distal end 86 can be inflated by an inflation fluid (e.g., air, saline, blood) to urge the prosthesis 10 and its support 20 radially outward. The support 20 can be configured (e.g., as an inelastically deformable material) as to remain in its expanded condition following the inflation of the balloon 90.

An appropriately sized second heart valve prosthesis 80 can then be implanted (at least partially) within the expanded heart valve prosthesis 10, such as shown in FIG. 9. By way of example, in FIG. 9, the heart valve prosthesis 80 is implanted through the pulmonary artery 84 using a low-invasive, closed heart procedure, such as shown and described in the above incorporated co-pending U.S. patent application Ser. No. 10/266,380. For example, an incision 92 is made in the pulmonary artery at a location that provides for a line-of-sight from the incision to the implantation site within the interior of the previously implanted (and expanded) prosthesis 10. A barrel 94 of an implanter device 96 is advanced through the incision 92 to position a distal end 98 of the barrel adjacent the implantation site. The second prosthesis 80 is ejected from the barrel, such as by causing a plunger to urge the prosthesis out of the distal end. The prosthesis 80 can be expanded (either manually or automatically, if self-expanding) to a desired diameter at the implantation site. The second prosthesis 80 can include spikes that secure the prosthesis at a desired position so as to mitigate axial and rotational movement of the prosthesis relative to the previously implanted prosthesis. Additional sutures or other means (e.g., clips, hooks) can be used to help secure the prosthesis at the implantation site. After the valve has been secured, the implanter can be removed and the incision closed.

Those skilled in the art will appreciate other approaches that can be used to implant the second heart valve prosthesis 80, which approaches can vary depending on the type of valve being implanted. The second heart valve prosthesis 80 can be the same type of prosthesis (e.g., such as the same type as the valve 12 of FIG. 1) or the second heart valve prosthesis can be a different type of valve.

What have been described above are examples of the invention. It is, of course, not possible to describe every conceivable combination of components or methodologies for purposes of describing the invention, but one of ordinary skill in the art will recognize that many further combinations and permutations of the present invention are possible. Accordingly, the invention is intended to embrace all such alterations, modifications and variations that fall within the spirit and scope of the appended claims.

What is claimed is:

1. A heart valve prosthesis comprising:
a generally cylindrical support structure configured to expand from a first cross-sectional dimension to a second cross-sectional dimension, which is larger than the first cross-sectional dimension;
a valve attached to the support structure to provide for substantially unidirectional flow of blood through the heart valve prosthesis in a direction from an inflow end to an outflow end thereof; and
a member operatively connected with the heart valve prosthesis to maintain temporarily the support structure at a cross-sectional dimension that is smaller than the second cross-sectional dimension, the member comprising a biodegradable material whereby, after being implanted for a substantially predetermined time period, the biodegradable material breaks down to permit the support structure to expand toward the second cross-sectional dimension.

2. The heart valve prosthesis of claim 1, wherein the biodegradable material further comprises a natural material.

3. The heart valve prosthesis of claim 1, wherein the biodegradable material further comprises a synthetic material.

4. The heart valve prosthesis of claim 1, wherein the biodegradable material further comprises at least one of a natural material and a synthetic material.

5. The heart valve prosthesis of claim 1, wherein the substantially predetermined time period is at least about four weeks.

6. The heart valve prosthesis of claim 1, wherein the substantially predetermined time period is at least about two months.

7. The heart valve prosthesis of claim 1, wherein the support structure comprises a self-expanding support structure.

8. An expandable type of heart valve prosthesis comprising:
means for retaining the heart valve prosthesis substantially at a first cross-sectional dimension that is smaller than a second expanded cross-sectional dimension, and for breaking down sufficiently to permit the support structure to expand toward the second expanded cross-sectional dimension after being implanted for a substantially predetermined time period; and
means for expanding the heart valve prosthesis from the first cross-sectional dimension toward the second expanded cross-sectional dimension.

9. The heart valve prosthesis of claim 8, wherein the means for retaining further comprises at least one suture.

10. The heart valve prosthesis of claim 8, wherein the means for retaining further comprises at least one web of natural material.

11. The heart valve prosthesis of claim 8, wherein the means for retaining further comprises at least one sheet of a pliant material and at least one suture to hold the at least one sheet about the heart valve prosthesis.

12. The heart valve prosthesis of claim 8, wherein the means for retaining further comprises a biodegradable natural material.

13. The heart valve prosthesis of claim 8, wherein the means for retaining further comprises a biodegradable synthetic material.

14. The heart valve prosthesis of claim 8, wherein the means for retaining further comprises at least one of a biodegradable natural material and a biodegradable synthetic material.

15. The heart valve prosthesis of claim 8, wherein the substantially predetermined time period is at least about four weeks.

16. The heart valve prosthesis of claim 8, wherein the substantially predetermined time period is at least about two months.

17. The heart valve prosthesis of claim 8, wherein the support structure comprises a self-expanding support structure.

18. A method comprising:
providing a heart valve prosthesis configured to permit post-implantation size change from a first cross-sectional dimension to a second cross-sectional dimension, which is larger than the first cross-sectional dimension, the heart valve prosthesis being configured to maintain temporarily the first cross-sectional dimension for a substantially predetermined period of time after being implanted;
implanting the heart valve prosthesis at an implantation site in a patient's heart to replace a function of a patient's native valve; and
after the occurrence of the substantially predetermined period of time after being implanted, the heart valve prosthesis expanding from the first cross-sectional dimension toward the second cross-sectional dimension commensurate with growth of the patient's heart at the implantation site.

19. The method of claim 18, wherein the expansion of the heart valve prosthesis occurs automatically.

20. The method of claim 18, wherein the heart valve prosthesis is a first heart valve prosthesis, the method further comprising:
manually expanding the first heart valve prosthesis to or beyond the second cross-sectional dimension; and
implanting a second heart valve prosthesis at least partially within an interior of the expanded first heart valve prosthesis.
